(19) United States
(12) Patent Application Publication
(10) Pub. No.: US 2022/0249008 A1
(43) Pub. Date: Aug. 11, 2022

(54) METHOD AND DEVICE FOR LOCALIZING EPILEPTOGENIC ZONES

(71) Applicant: The Johns Hopkins University, Baltimore, MD (US)

(72) Inventors: Adam LI, Thousand Oaks, CA (US); Sridevi V. SARMA, McLean, VA (US)

(73) Assignee: The Johns Hopkins University, Baltimore, MD (US)

(21) Appl. No.: 17/597,211

(22) PCT Filed: Jul. 17, 2020
(86) PCT No.: PCT/US2020/042570

§ 371 Date: Dec. 29, 2021

Related U.S. Application Data
(60) Provisional application No. 62/876,529, filed on Jul. 19, 2019.

(51) Int. Cl. A61B 5/372; G16H 50/20; G16H 50/50; G16H 40/60

(52) U.S. Cl. CPC A61B 5/372; G16H 40/60; G16H 50/50; G16H 50/20

(57) ABSTRACT

A device may receive electroencephalography data relating to one or more cerebral regions. The device may generate, based on the electroencephalography data, a cortical stimulation mapping model of the one or more cerebral regions, wherein the cortical stimulation mapping model includes one or more virtual inputs and one or more virtual outputs corresponding to the one or more cerebral regions. The device may apply a virtual impulse to the one or more virtual inputs. The device may determine a virtual after-discharge from the one or more virtual outputs, wherein the virtual after-discharge includes information relating to an electrical response to the virtual impulse. The device may generate, based on the virtual after-discharge, an index that maps a magnitude of the virtual after-discharge to the one or more cerebral regions. The device may cause an action to be performed based on the index.

### CROSS-REFERENCE TO RELATED APPLICATION

This Patent Application claims priority to U.S. Provisional Patent Application No. 62/876,529, filed on Jul. 19, 2019, and entitled “METHOD AND DEVICE FOR LOCALIZING EPILEPTOGENIC ZONES,” the content of which is incorporated by reference herein in its entirety.

### GOVERNMENT LICENSE RIGHTS

This invention was made with U.S. Government support under grant R21NS103113, awarded by the National Institute of Health (NIH) and grant DGE-1746891, awarded by the National Science Foundation. The U.S. Government has certain rights in the invention.

### BACKGROUND

Cortical stimulation mapping is a type of electrocorticography that involves a surgically invasive procedure and serves to localize functions of specific cerebral regions of a brain through direct electrical stimulation of a cerebral cortex. Cortical stimulation mapping may allow clinicians to analyze the brain and study relationships between cortical structure and systemic function. Cortical stimulation mapping may be used for a number of clinical and therapeutic applications, and enable pre-surgical mapping of motor cortex and language areas to prevent unnecessary functional damage. Cortical stimulation mapping may also be used to analyze cerebral regions and identify epileptogenic cerebral regions or epileptogenic zones for treatment of epilepsy patients.

### SUMMARY

According to some implementations, a method may include receiving, by a device, electroencephalography data relating to one or more cerebral regions of a cerebral cortex; generating, by the device, and based on the electroencephalography data, a cortical stimulation mapping model of the one or more cerebral regions, wherein the cortical stimulation mapping model includes one or more virtual inputs and one or more virtual outputs corresponding to the one or more cerebral regions; applying, by the device, a virtual impulse to the one or more virtual inputs of the cortical stimulation mapping model; determining, by the device, a virtual after-discharge from the one or more virtual outputs of the cortical stimulation mapping model, wherein the virtual after-discharge includes information relating to an electrical response to the virtual impulse; generating, by the device, an index based on the virtual after-discharge, wherein the index maps a magnitude of the virtual after-discharge to the one or more cerebral regions; and causing, by the device, an action to be performed based on the index.

According to some implementations, a device may include one or more memories; and one or more processors, communicatively coupled to the one or more memories, configured to: receive electroencephalography data relating to one or more cerebral regions of a cerebral cortex; generate a cortical stimulation mapping model of the one or more cerebral regions based on the electroencephalography data, wherein the cortical stimulation mapping model includes one or more virtual inputs and one or more virtual outputs corresponding to the one or more cerebral regions; apply a virtual impulse to the one or more virtual inputs of the cortical stimulation mapping model; determine a virtual after-discharge from the one or more virtual outputs of the cortical stimulation mapping model, wherein the virtual after-discharge includes information relating to an electrical response to the virtual impulse; generate a heat map based on the virtual after-discharge, wherein the heat map visually maps a magnitude of the virtual after-discharge to the one or more cerebral regions; identify an epileptogenic zone based on the heat map; and cause an action to be performed based on the epileptogenic zone.

According to some implementations, a non-transitory computer-readable medium may store one or more instructions. The one or more instructions, when executed by one or more processors, may cause the one or more processors to: receive electroencephalography data relating to a plurality of cerebral regions of a cerebral cortex; generate a cortical stimulation mapping model of the plurality of cerebral regions based on the electroencephalography data, wherein the cortical stimulation mapping model includes a plurality of virtual inputs and a plurality of virtual outputs corresponding to the plurality of cerebral regions; apply a plurality of virtual impulses to the plurality of virtual inputs of the cortical stimulation mapping model; determine a plurality of virtual after-discharges from the plurality of virtual outputs of the cortical stimulation mapping model, wherein the plurality of virtual after-discharges includes information relating to respective electrical responses to the plurality of virtual impulses; generate a heat map based on the plurality of virtual after-discharges, wherein the heat map visually maps respective magnitudes of the plurality of virtual after-discharges to the plurality of cerebral regions; identify an epileptogenic zone based on the heat map; and cause an action to be performed based on the epileptogenic zone.

### BRIEF DESCRIPTION OF THE DRAWINGS

FIGS. 1A-1E are diagrams of one or more example implementations described herein.

FIG. 2 is a diagram of an example environment in which systems and/or methods described herein may be implemented.

FIG. 3 is a diagram of example components of one or more devices of FIG. 2.

FIG. 4 is a flow chart of an example process for localizing epileptogenic zones.

FIG. 5 is a flow chart of an example process for localizing epileptogenic zones.

FIG. 6 is a flow chart of an example process for localizing epileptogenic zones.

### DETAILED DESCRIPTION

The following detailed description of example implementations refers to the accompanying drawings. The same reference numbers in different drawings may identify the same or similar elements.

Cortical stimulation mapping is a surgically invasive clinical procedure that may be used to map eloquent cortex of a subject (e.g., an epilepsy patient). During cortical stimulation mapping, electrodes may be placed directly onto a cerebral cortex of a subject to monitor and record electrical activity from different cerebral regions of the cerebral cortex (e.g., motor regions, essential somatosensory regions, and/or the like). In particular, the electrodes may apply electrical stimulation to the cerebral regions, and monitor for electrical responses to the electrical stimulation (e.g., after-discharges). The after-discharges may be recorded in a form of electroencephalogram (EEG) data (e.g., intracranial electroencephalography (iEEG) data, electrocorticography (ECoG) data, stereo-electroencephalogram (SEEG) data, and/or the like). The after-discharges can reveal abnormal or unstable cerebral regions that are more prone to spontaneous seizures, which can further be indicative of an epileptogenic zone (e.g., a source of the spontaneous seizures). Based on the epileptogenic zone, clinicians may be able to identify and target certain cerebral regions for treatment (e.g., surgical resection, laser ablation, and/or the like). Although cortical stimulation mapping may be helpful in treating epilepsy, there is room for improvement.

In some cases, an epileptogenic zone may extend into cerebral regions that are beyond a testable surface of a cerebral cortex and inaccessible via in-vivo cortical stimulation mapping. Although a clinician may prefer to more thoroughly test additional cerebral regions, doing so can be time-consuming and potentially harmful to a subject. In-vivo cortical stimulation mapping procedures thereby remain limited in testing capability. Furthermore, with such limitations, treatments performed based on in-vivo cortical stimulation mapping procedures may produce unsuccessful results (e.g., an epilepsy patient may continue to have seizures after removal of a suspected epileptogenic zone), and may lead to additional surgical procedures. For example, if a subject continues to have seizures after treatment, the subject may undergo additional surgically invasive testing (e.g., additional in-vivo cortical stimulation mapping procedures) and/or additional surgical treatments in order to successfully treat the epilepsy. This can prolong treatment and add more time in an operating room, which can further introduce unwanted risks, substantial costs, and resources associated with the prolonged treatment.

Some implementations described herein provide a localization platform that may assist clinicians in mapping cerebral regions and localizing epileptogenic zones with fewer constraints. The localization platform may receive EEG data relating to the cerebral regions, generate a cortical stimulation mapping model of the cerebral regions based on the EEG data, apply virtual impulses to virtual inputs of the cortical stimulation mapping model, determine virtual after-discharges from virtual outputs of the cortical stimulation mapping model, generate an index of the cerebral regions based on the virtual after-discharges, and cause an action to be performed based on the index. In some implementations, the localization platform may generate the index in the form of a heat map that identifies unstable cerebral regions based on the virtual after-discharges, identify an epileptogenic zone based on the index, and/or provide other information to a clinician. In some implementations, the localization platform may generate a visual model of the cerebral regions that a clinician can use to more easily localize an epileptogenic zone. In some implementations, the localization platform may compare a clinically annotated epileptogenic zone with the index, and determine a treatment success rate based on the clinically annotated epileptogenic zone.

In this way, the localization platform may enable clinicians to more thoroughly perform cortical stimulation mapping and more accurately identify an epileptogenic zone without additional costs, time, resources, and risks associated with in-vivo cortical stimulation mapping procedures. For instance, because tests are conducted using a virtual model rather than on an actual subject (e.g., an epilepsy patient), clinicians are able to study a wider range of cerebral regions in less time and without exposing the subject to risks associated with surgically invasive procedures. Since clinicians are able to more confidently identify epileptogenic zones in a first instance, the localization platform enables clinicians to perform more effective treatment with better success rates. The localization platform may thereby reduce the need for repeated surgical procedures (e.g., performing repeated in-vivo cortical stimulation mapping procedures, repeated surgical treatment, and/or the like), and may further conserve computational resources, network resources, and/or power resources needed to operate surgical equipment and/or other surgical facilities.

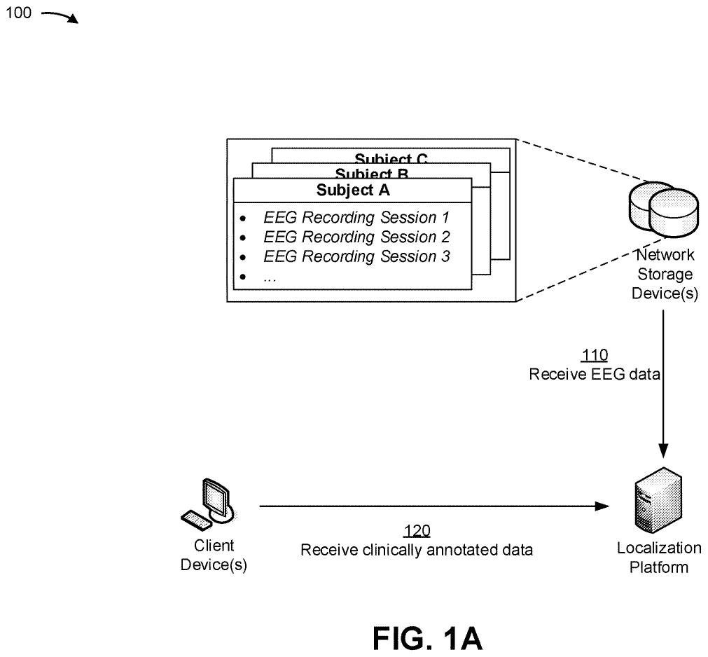

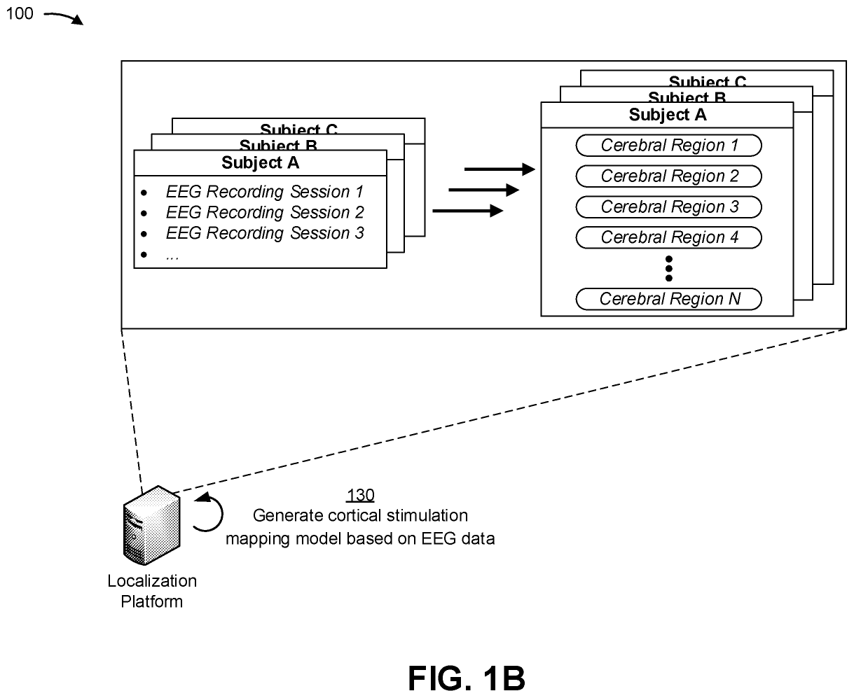

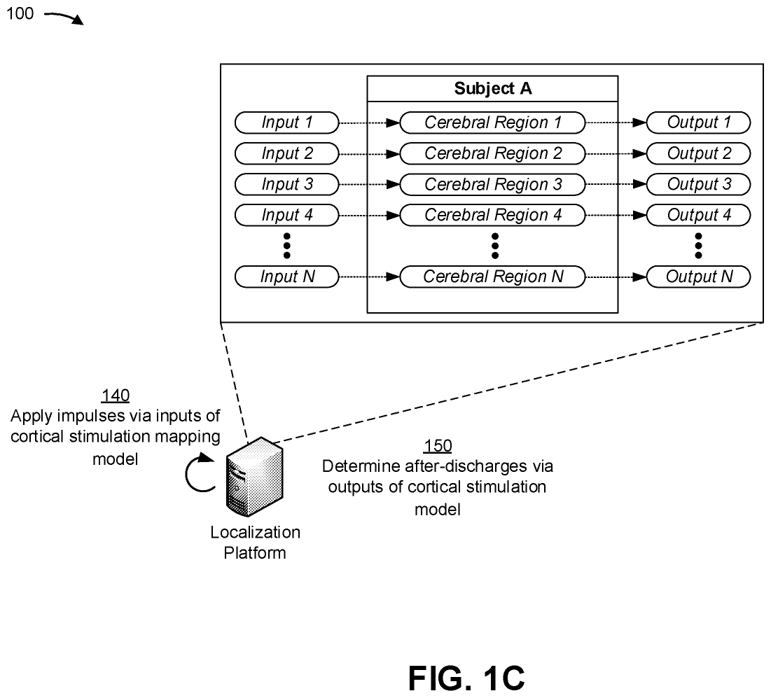

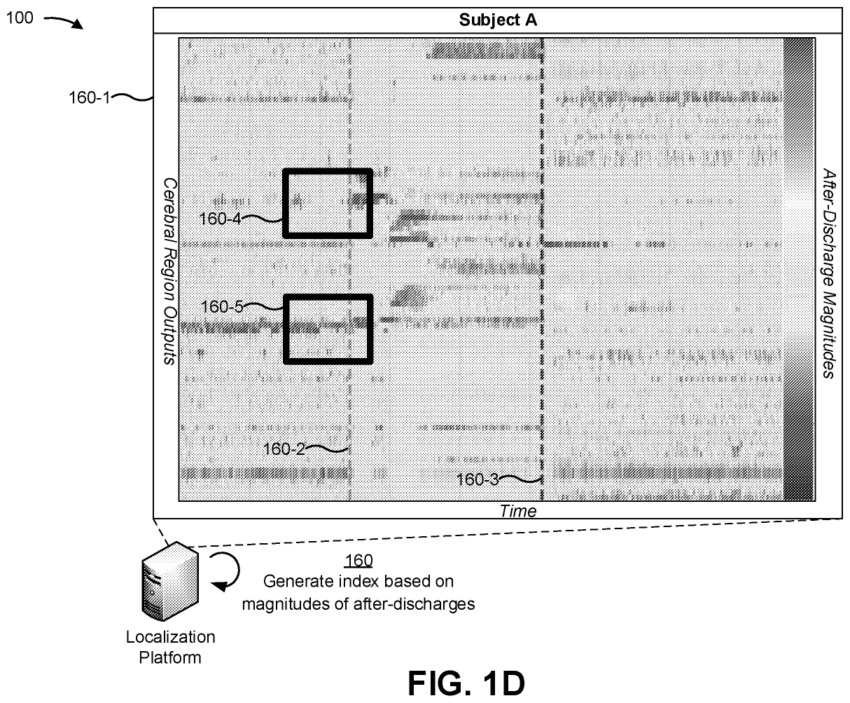

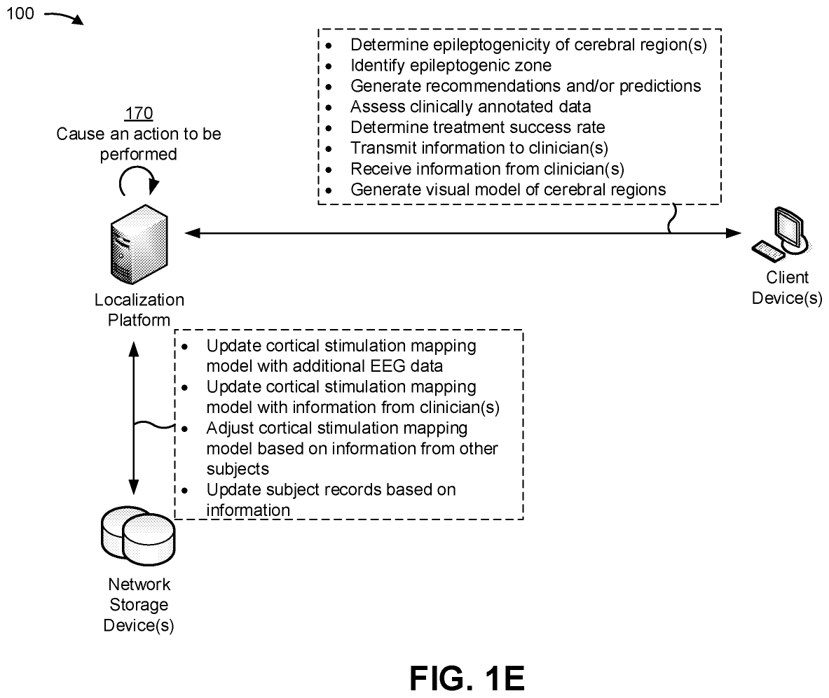

FIGS. 1A-1E are diagrams of one or more example implementations 100 described herein. As shown in FIGS. 1A-1E, the example implementation(s) 100 may include a localization platform, a network storage device, and a client device. FIGS. 1A-1E present one or more functions that may be performed by the localization platform to map cerebral regions of a cerebral cortex and localize an epileptogenic cerebral region and/or an epileptogenic zone. For example, the localization platform may receive EEG data relating to the cerebral regions, generate a cortical stimulation mapping model of the cerebral regions based on the EEG data, apply virtual impulses to virtual inputs of the cortical stimulation mapping model, determine virtual after-discharges from virtual outputs of the cortical stimulation mapping model, generate an index of the cerebral regions based on the virtual after-discharges, and cause an action to be performed based on the index. In some implementations, one or more of the functions, described as being performed by the localization platform, may be performed by another device, such as the network storage device, the client device, and/or the like.

In some implementations, the localization platform may be used in association with a localization service that is supported by the network storage device. For example, the localization service may be used by a user (e.g., a clinician, a surgeon, a nurse, another medical professional, and/or another subscriber) to analyze EEG data, access a virtual cortical stimulation mapping model based on the EEG data, perform virtual cortical stimulation mapping to identify unstable cerebral regions and/or an epileptogenic zone, access a virtual model of the cerebral regions, and/or the like. The localization service may provide features, such as providing a heat map that identifies unstable and potentially epileptogenic cerebral regions for a user, identifying a potential epileptogenic zone for the user, comparing a clinically annotated epileptogenic zone with an epileptogenic zone identified by the virtual cortical stimulation mapping model, determining a treatment success rate for the user, and/or providing other useful information to the user. A user may access the localization service using a client device (e.g., a computer, a smart phone, a mobile device, and/or the like) that is connected to the localization platform over a wired connection and/or a wireless connection.

As shown in FIG. 1A, and by reference number 110, the localization platform may receive EEG data from a network storage device. For example, a network storage device may store EEG data collected from a subject (e.g., an epilepsy patient) and previously recorded by a clinician. The EEG data may include one or more recording sessions containing information relating to electrical activity of cerebral regions of a cerebral cortex of a subject. For example, a recording session of the EEG data may include electrical activity that is detected using electrodes implanted or placed directly on the cerebral cortex of the subject, and recorded as a waveform having an amplitude and/or a frequency corresponding to the electrical activity. The EEG data may include electrical activity corresponding to normal brain activity, electrical responses to stimuli initiated by a clinician, electrical activity associated with a seizure event, and/or the like. In some examples, the EEG data may be provided in a form of iEEG data, ECoG data, SEEG data, and/or the like. The localization platform may receive EEG data associated with a single subject or multiple subjects from the network storage device.

As further shown in FIG. 1A, and by reference number 120, the localization platform may receive clinically annotated data from a client device. For example, the clinically annotated data may include information identifying a cerebral region, a subset of a cerebral region, a superset of a cerebral region, and/or a set of cerebral regions that a clinician suspects as being epileptogenic (e.g., based on prior tests and/or analyses). The localization platform may receive the clinically annotated data from the clinician via a user interface of a client device (e.g., via access to a localization service provided by the localization platform). For example, the localization service may provide a service that enables the clinician to compare the clinically annotated cerebral regions with corresponding information provided by the localization platform, estimate a success rate of treating the clinically annotated cerebral regions, and/or the like. In some examples, the localization platform may receive the clinically annotated data from another type of user (e.g., a surgeon, a nurse, and/or another medical professional) via the client device. In some examples, the localization platform may receive other types of information from the client device that the localization platform may use to localize an epileptogenic zone.

As shown in FIG. 1B, and by reference number 130, the localization platform may generate a cortical stimulation mapping model based on the EEG data. For example, the localization platform may use the EEG data to determine historic electrical activity recorded from cerebral regions of a subject, and construct a virtual model of the cerebral regions (e.g., an in-silico cortical stimulation mapping model) based on the historic electrical activity. The localization platform may generate the cortical stimulation mapping model with virtual inputs and virtual outputs associated with the cerebral regions (e.g., simulating electrodes used during in-vivo cortical stimulation mapping procedures) that respond to virtual stimuli in a manner that is consistent with the historic electrical activity obtained from the EEG data. In some examples, the localization platform may generate a cortical stimulation mapping model for a subject based on the EEG data associated with the subject. Additionally, or alternatively, the localization platform may generate a cortical stimulation mapping model for a subject that incorporates information obtained from EEG data associated with another subject and/or information obtained from another cortical stimulation mapping model.

In some implementations, the localization platform may generate the cortical stimulation mapping model based on a linear time varying network of the EEG data. The localization platform may construct the linear time varying network by generating discrete linear time-invariant systems of the EEG data, and concatenating a sequence of the discrete linear time-invariant systems of the EEG data. For example, a state evolution of a discrete linear time-invariant system may be expressed as,

x(t+1)=ƒᵢ(x(t)) (1)

where x(t) corresponds to an element of a state vector, ƒ corresponds to a well-behaved function, t corresponds to a unit of time, and i corresponds to a time variant. For example, a linear model of Equation 1 takes on the form:

x(t+1)=Aᵢ(x(t)) (2)

where Aᵢ corresponds to a state transition matrix. An element of the state vector may include information relating to electrical activity of a cerebral region within a network of cerebral regions associated with the EEG data. An element of the state transition matrix may include information relating to functional dynamics of a cerebral region, functional effects of a cerebral region on another cerebral region, and/or other information interrelating the electrical activity observed between the cerebral regions of the network associated with the EEG data. In some examples, the state transition matrix may be defined with one or more dimensions that correspond to a cumulative functional effect of the network on a cerebral region, a functional effect of a cerebral region on the network, and/or the like.

In some implementations, the localization platform may use a least squares analysis (e.g., a sliding window least-squares approach and/or the like) to concatenate the discrete linear time-invariant systems and generate the linear time variant network, which may be expressed as

D=A₁,A₂,A₃, . . . , A_{W} (3)

where D corresponds to the linear time variant network, Aᵢ corresponds to elements of the state transition matrix, and W corresponds to a number of sliding windows used. Using the linear time variant network, the localization platform may be able to generate a cortical stimulation mapping model that can be used to test the cerebral regions of a subject within a virtual environment and without constraints and/or risks associated with surgically invasive procedures. The cortical stimulation mapping model may be stored within the localization platform, the client device, and/or the network storage device. In some examples, the cortical stimulation mapping model may be generated by the client device and/or the network storage device. In some examples, another device (e.g., a server device, a cloud computing device, and/or the like) may generate the cortical stimulation mapping model and provide the cortical stimulation mapping model for use by the localization platform, the client device, and/or the network storage device. Additionally, or alternatively, the localization platform may generate the cortical stimulation mapping model for use by another device (e.g., a server device, a cloud computing device, and/or the like).

As shown in FIG. 1C, and by reference number 140, the localization platform may apply a virtual impulse via a virtual input of the cortical stimulation mapping model. For example, the localization platform may virtually stimulate different cerebral regions of the cortical stimulation mapping model to observe electrical responses (e.g., virtual after-discharges) to the stimuli. In some examples, the localization platform may apply a virtual impulse according to a discrete time system that may be expressed as

Δⱼ=[0, . . . , 0,1,0, . . . , 0] (4)

where Δ corresponds to a unit impulse vector that is applied to the cortical stimulation mapping model, and j corresponds to a virtual input variant. For example, the unit impulse vector may be configured to cause a change in a magnitude of an electrical response of a cerebral region that is detectable as a virtual after-discharge from a corresponding virtual output. The localization platform may stimulate individual virtual inputs of the cortical stimulation mapping model (e.g., corresponding to different cerebral regions) using a common virtual impulse and/or using different virtual impulses (e.g., defined by distinct unit impulse vectors). In some examples, the localization platform may apply the virtual impulse to the individual virtual inputs simultaneously or at different times.

As further shown in FIG. 1C, and by reference number 150, the localization platform may determine a virtual after-discharge via a virtual output of the cortical stimulation mapping model. For example, the localization platform may observe a magnitude of the virtual after-discharge of a cerebral region of the cortical stimulation mapping model that results from the virtual impulse applied to a corresponding virtual input of the cerebral region. In some examples, the localization platform may determine a magnitude of a virtual after-discharge associated with a cerebral region based on an expression, such as

xⱼ(t+1)=Aᵢxⱼ(t)+Δⱼ(t) (5)

where Δ corresponds to a unit impulse vector (e.g., expression (3) above), x(t) corresponds to an electrical response (e.g., a virtual after-discharge of a cerebral region) to the unit impulse vector, A corresponds to a discrete linear time-invariant system from the linear time variant network, t corresponds to a unit of time, i corresponds to a time variant, and j corresponds to a virtual output variant. The localization platform may determine the virtual after-discharge of individual virtual outputs of the cortical stimulation mapping model (e.g., corresponding to different cerebral regions) simultaneously or at different times.

As shown in FIG. 1D, and by reference number 160, the localization platform may generate an index of respective virtual after-discharges determined from corresponding virtual outputs of the cortical stimulation mapping model. For example, the index may map respective magnitudes of the virtual after-discharges to corresponding cerebral regions in a manner configured to facilitate a comparison of electrical responses across the cerebral regions. In some examples, the localization platform may generate the index as a heat map 160-1 based on the virtual after-discharges. For example, the localization platform may apply a vector norm (e.g., an L2 norm and/or the like) to the virtual after-discharges (e.g., expression (4) above) of the virtual outputs to determine respective magnitudes of the virtual after-discharges. In some examples, the localization platform may normalize (e.g., using Z-normalization and/or the like) the magnitudes of the virtual after-discharges across the cerebral regions to provide a cumulative heat map system defined by

$${R}_{j i} = \frac{{R}_{ji} - μ ( {R}_{ji} )}{σ ( {R}_{ji} )} \qquad ( 6 )$$

where R corresponds to heat map 160-1, μ corresponds to a mean of the virtual after-discharges across the virtual outputs within a window of time, σ corresponds to a standard deviation of the virtual after-discharges across the virtual outputs within a window of time, i corresponds to a time variant, and j corresponds to a virtual output variant.

In some implementations, the localization platform may generate heat map 160-1 to include indications (e.g., color-coded indications and/or the like) of the respective magnitudes of the virtual after-discharges corresponding to the cerebral regions. As shown for the example in FIG. 1D, heat map 160-1 may visually map the respective magnitudes of the virtual after-discharges as a function of time, and use darker tones to indicate virtual after-discharges with greater magnitudes (e.g., corresponding to irregular electrical responses that may be suggestive of epileptogenic cerebral regions). In some examples, the localization platform may provide an indication of an onset (e.g., start time 160-2) and/or an offset (e.g., end time 160-3) of a virtual impulse applied to the cortical stimulation mapping model. In some examples, the localization platform may include annotations of irregular areas (e.g., area 160-4, area 160-5, and/or the like) of heat map 160-1 to indicate virtual after-discharges having relatively greater magnitudes (e.g., corresponding to irregular electrical responses that may be suggestive of epileptogenic cerebral regions). The localization platform may identify irregular areas 160-4, 160-5 in relation to the virtual impulse (e.g., before start time 160-2, at start time 160-2, after start time 160-2, and/or at a time between start time 160-2 and end time 160-3).

As shown in FIG. 1E, and by reference number 170, the localization platform may cause an action to be performed based on the index. In some examples, localization platform may compare a magnitude of a virtual after-discharge with a threshold magnitude, and identify an epileptogenic zone based on a comparison between the magnitude of the virtual after-discharge and the threshold magnitude. For example, the localization platform may determine that the epileptogenic zone includes a cerebral region if a magnitude of a virtual after-discharge of the cerebral region satisfies the threshold magnitude. Correspondingly, the localization platform may determine that the epileptogenic zone does not include a cerebral region if a magnitude of a virtual after-discharge of the cerebral region does not satisfy the threshold magnitude. The threshold magnitude may be determined based on an average of respective magnitudes of virtual after-discharges across the cerebral regions modeled by the cortical stimulation mapping model. In some examples, the localization platform may similarly identify epileptogenicity of a subset of a cerebral region, a superset of a cerebral region, and/or a set of cerebral regions.

In some implementations, the localization platform may determine an epileptogenicity of a cerebral region based on the index (e.g., based on a threshold comparison, a color code of heat map 160-1, annotations of irregular areas 160-4, 160-5, and/or the like), generate a recommendation for a clinician based on the epileptogenicity, and transmit the recommendation to a clinician and/or another user (e.g., a surgeon, a nurse, and/or another medical professional) via a user interface of a client device (e.g., via a localization service provided by the localization platform). For example, the recommendation may identify an epileptogenicity of a cerebral region, a subset of a cerebral region, a superset of a cerebral region, and/or a set of cerebral regions, identify an epileptogenic zone based on the epileptogenicity, and/or other information that may assist the clinician in identifying and/or treating a subject (e.g., an epilepsy patient). In some examples, the localization platform may receive feedback and/or information from the clinician via the client device that the localization platform may use to provide more accurate recommendations.

In some implementations, the localization platform may receive clinically annotated data relating to a clinically annotated epileptogenic zone (e.g., one or more cerebral regions that have been identified by a clinician as being epileptogenic) from a client device. The localization platform may compare the clinically annotated epileptogenic zone to the index (e.g., heat map 160-1), and determine a treatment success rate based on the clinically annotated epileptogenic zone. For example, the localization platform may determine a consistency between the clinically annotated epileptogenic zone and cerebral regions identified by the index as being epileptogenic, and predict a likelihood that treatment of the clinically annotated epileptogenic zone will be successful based on the consistency. If the clinically annotated epileptogenic zone identifies all of the epileptogenic cerebral regions identified by the index, the localization platform may determine a high treatment success rate. If the clinically annotated epileptogenic zone identifies a cerebral region that is not identified by the index as epileptogenic and/or omits a cerebral region that is identified by the index as epileptogenic, the localization platform may determine a low treatment success rate. The treatment success rate may be determined as a percentage value, a score, a rating, a ratio, and/or another metric. In some examples, the localization platform may generate a recommendation and/or a prediction for the clinician based on the treatment success rate, and transmit the recommendation and/or the prediction to the clinician via the client device.

In some implementations, the localization platform may predict a treatment success rate based on an impulse response ratio calculated using heat map 160-1. For example, the localization platform may analyze a sample of heat map 160-1 corresponding to a virtual after-discharge that is directly in response to a virtual impulse (e.g., from a time before start time 160-2 to a time after end time 160-3 and/or the like), adjust a sample size (e.g., up-sample or down-sample the virtual after-discharge) for consistency across different cerebral regions and/or different subjects, and determine the impulse response ratio based on the sample. The localization platform may partition a heat map system (e.g., expression (5) above) into a clinically annotated heat map and an unannotated heat map, and determine the impulse response ratio based on a comparison between the clinically annotated heat map and the unannotated heat map according to

$$impulse response ratio = \frac{\frac{1}{W} {Σ}_{j = 1}^{W} {Σ}_{i = 1}^{{N}_{cez}} {R}_{ji}^{′}}{\frac{1}{W} {Σ}_{j = 1}^{W} {Σ}_{i = 1}^{{N}_{oez}} {R}_{ji}^{′}} \qquad ( 7 )$$

where W corresponds to a number of sliding windows used for heat map 160-1, N_{cez} corresponds to a number of clinically annotated cerebral regions, N_{oez} corresponds to a number of unannotated cerebral regions, R′ corresponds to a respective partition of heat map 160-1, i corresponds to a time variant, and j corresponds to a virtual output variant. The localization platform may determine the treatment success rate based on the impulse response ratio (e.g., determine a higher treatment success rate for a higher impulse response ratio, and a lower treatment success rate for a lower impulse response ratio).

In some implementations, the localization platform may generate a visual model based on the index. For example, the localization platform may generate a two-dimensional virtual model and/or a three-dimensional virtual model of one or more cerebral regions and/or a cerebral cortex of a subject, generate a graphical representation of an epileptogenic zone and/or a cerebral region identified by the index as being epileptogenic, and overlay the graphical representation of the epileptogenic zone and/or the epileptogenic cerebral region on the visual model at a corresponding location within the visual model. The graphical representation of the epileptogenic zone and/or the epileptogenic cerebral region may be color-coded or otherwise indicative of epileptogenicity. The localization platform may transmit the visual model to a clinician and/or another user via a user interface of a client device. In some examples, the localization platform may enable the clinician to perform virtual cortical stimulation mapping via the virtual model. For example, the visual model may include graphical representations of electrodes corresponding to the virtual inputs and/or the virtual outputs of the cortical stimulation mapping model and simulate an in-vivo cortical stimulation mapping environment for the clinician. Additionally, or alternatively, the localization platform may enable the clinician to view, manipulate, edit, and/or update the virtual model and/or an associated heat map 160-1 via the user interface of the client device.

In some implementations, the localization platform may update the cortical stimulation mapping model with additional information that may be available on and received from the network storage device. For example, the localization platform may update the cortical stimulation mapping model with additional EEG data and/or other information relating to a subject that may be provided by a clinician and/or another user. In some examples, the localization platform may update the cortical stimulation mapping model using information obtained from other subjects (e.g., trends, patterns, and/or relationships between cerebral activity and epileptogenicity) that may be used to improve an accuracy of the cortical stimulation mapping model. Additionally, or alternatively, the localization platform may update a record of a subject that is stored on the network storage device with information determined by the localization platform (e.g., a previously unannotated cerebral region that was found to be epileptogenic, a previously annotated cerebral region that was found to be not epileptogenic, and/or the like).

In this way, the localization platform may enable clinicians to more thoroughly perform cortical stimulation mapping and more accurately identify an epileptogenic zone without additional costs, time, resources, and risks associated with in-vivo cortical stimulation mapping procedures. For instance, because tests are conducted using a virtual model rather than on an actual subject (e.g., an epilepsy patient), clinicians are able to study a wider range of cerebral regions in less time and without exposing the subject to risks associated with surgically invasive procedures. Since clinicians are able to more confidently identify epileptogenic zones in a first instance, the localization platform enables clinicians to perform more effective treatment with better success rates. The localization platform may thereby reduce the need for repeated surgical procedures (e.g., performing repeated in-vivo cortical stimulation mapping procedures, repeated surgical treatment, and/or the like), and may further conserve computational resources, network resources, and/or power resources needed to operate surgical equipment and/or other surgical facilities.

As indicated above, FIGS. 1A-1E are provided as one or more examples. Other examples can differ from what is described with regard to FIGS. 1A-1E.

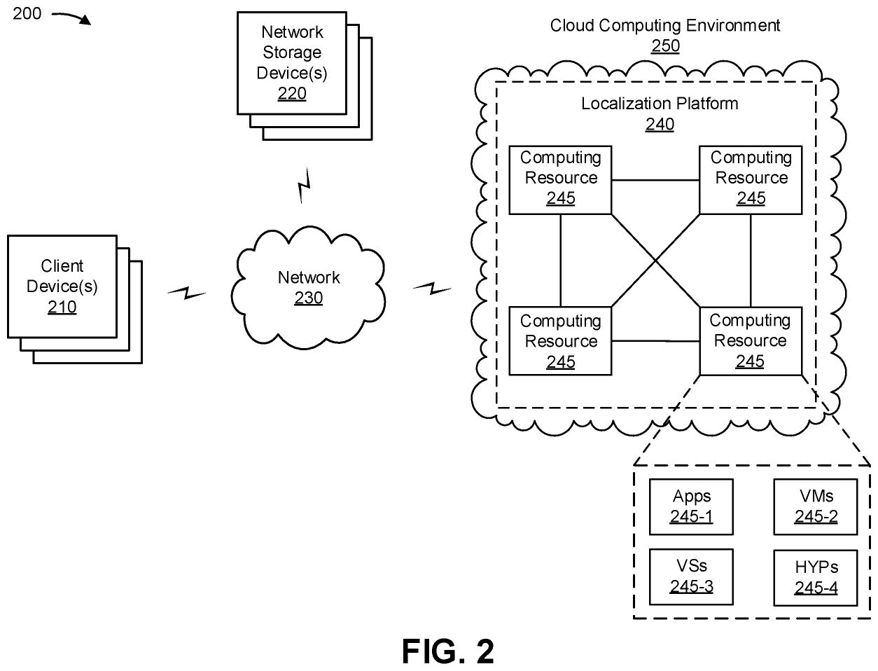

FIG. 2 is a diagram of an example environment 200 in which systems and/or methods, described herein, may be implemented. As shown in FIG. 2, environment 200 may include one or more client devices 210 (referred to herein individually as client device 210 and collectively as client devices 210), one or more network storage devices 220 (referred to herein individually as network storage device 220 and collectively as network storage devices 220), network 230, localization platform 240, computing resource 245, and cloud computing environment 250. Devices of environment 200 may interconnect via wired connections, wireless connections, or a combination of wired and wireless connections.

Client device 210 includes one or more devices capable of receiving, generating, storing, processing, and/or providing information associated with identifying an epileptogenic cerebral region and/or an epileptogenic zone of a cerebral cortex. For example, client device 210 may include a communication and/or computing device, such as a mobile phone (e.g., a smart phone, a radiotelephone, and/or the like), a laptop computer, a tablet computer, a handheld computer, a desktop computer, a wearable communication device (e.g., a smart wristwatch, a pair of smart eyeglasses, and/or the like), or a similar type of device.

Network storage device 220 includes one or more devices capable of storing, processing, and/or routing information. Network storage device 220 may include, for example, a server device, a device that stores a data structure, a device in a cloud computing environment or a data center, a device in a core network of a network operator, a network controller, and/or the like. In some implementations, network storage device 220 may include a communication interface that allows network storage device 220 to receive information from and/or transmit information to other devices in environment 200, such as client device 210, localization platform 240, and/or the like.

Network 230 includes one or more wired and/or wireless networks. For example, network 230 may include a cellular network (e.g., a long-term evolution (LTE) network, a code division multiple access (CDMA) network, a 2G network, a 3G network, a 4G network, a 5G network, a new radio (NR) network, another type of next generation network, and/or the like), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, or the like, and/or a combination of these or other types of networks.

Localization platform 240 includes one or more computing devices configured to provide a cortical stimulation mapping model that can be used to identify and localize epileptogenic cerebral regions and/or an epileptogenic zone of a cerebral cortex. In some implementations, localization platform 240 may receive EEG data relating to cerebral regions of the cerebral cortex, generate a cortical stimulation mapping model of the cerebral regions based on the EEG data, apply virtual impulses to virtual inputs of the cortical stimulation mapping model, determine virtual after-discharges from virtual outputs of the cortical stimulation mapping model, generate an index of the cerebral regions based on the virtual after-discharges, and cause an action to be performed based on the index. In some implementations, localization platform 240 may be designed to be modular such that certain software components may be swapped in or out depending on a particular need. As such, localization platform 240 may be easily and/or quickly reconfigured for different uses. In some implementations, localization platform 240 may receive information from and/or transmit information to client device 210, network storage device 220, and/or the like.

In some implementations, localization platform 240 may include a server device or a group of server devices. In some implementations, localization platform 240 may be hosted in cloud computing environment 250. Notably, while implementations described herein describe localization platform 240 as being hosted in cloud computing environment 250, in some implementations, localization platform 240 may be non-cloud-based or may be partially cloud-based.

Cloud computing environment 250 includes an environment that delivers computing as a service, whereby shared resources, services, and/or the like may be provided to client device 210, network storage device 220, and/or the like. Cloud computing environment 250 may provide computation, software, data access, storage, and/or other services that do not require end-user knowledge of a physical location and configuration of a system and/or a device that delivers the services. As shown, cloud computing environment 250 may include localization platform 240 and computing resource 245.

Computing resource 245 includes one or more personal computers, workstation computers, server devices, or another type of computation and/or communication device. In some implementations, computing resource 245 may host localization platform 240. The cloud resources may include compute instances executing in computing resource 245, storage devices provided in computing resource 245, data transfer devices provided by computing resource 245, and/or the like. In some implementations, computing resource 245 may communicate with other computing resources 245 via wired connections, wireless connections, or a combination of wired and wireless connections.

As further shown in FIG. 2, computing resource 245 may include a group of cloud resources, such as one or more applications (“APPs”) 245-1, one or more virtual machines (“VMs”) 245-2, virtualized storage (“VSs”) 245-3, one or more hypervisors (“HYPs”) 245-4, or the like.

Application 245-1 includes one or more software applications that may be provided to or accessed by client device 210. Application 245-1 may eliminate a need to install and execute the software applications on client device 210. For example, application 245-1 may include software associated with localization platform 240 and/or any other software capable of being provided via cloud computing environment 250. In some implementations, one application 245-1 may send/receive information to/from one or more other applications 245-1, via virtual machine 245-2.

Virtual machine 245-2 includes a software implementation of a machine (e.g., a computer) that executes programs like a physical machine. Virtual machine 245-2 may be either a system virtual machine or a process virtual machine, depending upon use and degree of correspondence to any real machine by virtual machine 245-2. A system virtual machine may provide a complete system platform that supports execution of a complete operating system (“OS”). A process virtual machine may execute a single program and may support a single process. In some implementations, virtual machine 245-2 may execute on behalf of a user (e.g., client device 210), and may manage infrastructure of cloud computing environment 250, such as data management, synchronization, or long-duration data transfers.

Virtualized storage 245-3 includes one or more storage systems and/or one or more devices that use virtualization techniques within the storage systems or devices of computing resource 245. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 245-4 provides hardware virtualization techniques that allow multiple operating systems (e.g., “guest operating systems”) to execute concurrently on a host computer, such as computing resource 245. Hypervisor 245-4 may present a virtual operating platform to the guest operating systems and may manage the execution of the guest operating systems. Multiple instances of a variety of operating systems may share virtualized hardware resources.

The number and arrangement of devices and networks shown in FIG. 2 are provided as one or more examples. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

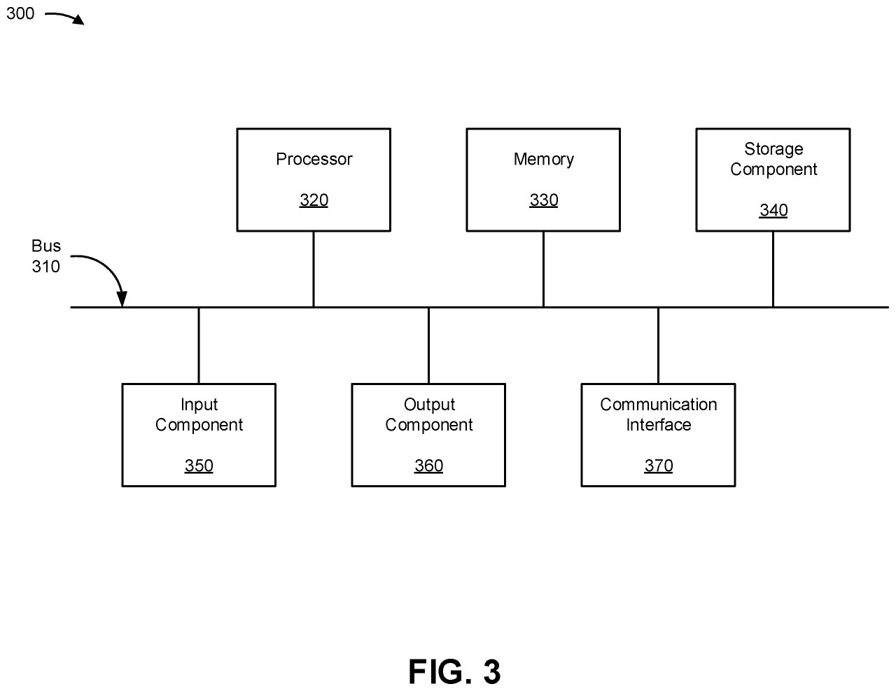

FIG. 3 is a diagram of example components of a device 300. Device 300 may correspond client device 210, network storage device 220, localization platform 240, and/or computing resource 245. In some implementations, client device 210, network storage device 220, localization platform 240, and/or computing resource 245 may include one or more devices 300 and/or one or more components of device 300. As shown in FIG. 3, device 300 may include a bus 310, a processor 320, a memory 330, a storage component 340, an input component 350, an output component 360, and a communication interface 370.

Bus 310 includes a component that permits communication among multiple components of device 300. Processor 320 is implemented in hardware, firmware, and/or a combination of hardware and software. Processor 320 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 320 includes one or more processors capable of being programmed to perform a function. Memory 330 includes a random access memory (RAM), a read only memory (ROM), and/or another type of dynamic or static storage device (e.g., a flash memory, a magnetic memory, and/or an optical memory) that stores information and/or instructions for use by processor 320.

Storage component 340 stores information and/or software related to the operation and use of device 300. For example, storage component 340 may include a hard disk (e.g., a magnetic disk, an optical disk, and/or a magneto-optic disk), a solid state drive (SSD), a compact disc (CD), a digital versatile disc (DVD), a floppy disk, a cartridge, a magnetic tape, and/or another type of non-transitory computer-readable medium, along with a corresponding drive.

Input component 350 includes a component that permits device 300 to receive information, such as via user input (e.g., a touch screen display, a keyboard, a keypad, a mouse, a button, a switch, and/or a microphone). Additionally, or alternatively, input component 350 may include a component for determining location (e.g., a global positioning system (GPS) component) and/or a sensor (e.g., an accelerometer, a gyroscope, an actuator, another type of positional or environmental sensor, and/or the like). Output component 360 includes a component that provides output information from device 300 (via, e.g., a display, a speaker, a haptic feedback component, an audio or visual indicator, and/or the like).

Communication interface 370 includes a transceiver-like component (e.g., a transceiver, a separate receiver, a separate transmitter, and/or the like) that enables device 300 to communicate with other devices, such as via a wired connection, a wireless connection, or a combination of wired and wireless connections. Communication interface 370 may permit device 300 to receive information from another device and/or provide information to another device. For example, communication interface 370 may include an Ethernet interface, an optical interface, a coaxial interface, an infrared interface, a radio frequency (RF) interface, a universal serial bus (USB) interface, a wireless local area network interface, a cellular network interface, and/or the like.

Device 300 may perform one or more processes described herein. Device 300 may perform these processes based on processor 320 executing software instructions stored by a non-transitory computer-readable medium, such as memory 330 and/or storage component 340. As used herein, the term “computer-readable medium” refers to a non-transitory memory device. A memory device includes memory space within a single physical storage device or memory space spread across multiple physical storage devices.

Software instructions may be read into memory 330 and/or storage component 340 from another computer-readable medium or from another device via communication interface 370. When executed, software instructions stored in memory 330 and/or storage component 340 may cause processor 320 to perform one or more processes described herein. Additionally, or alternatively, hardware circuitry may be used in place of or in combination with software instructions to perform one or more processes described herein. Thus, implementations described herein are not limited to any specific combination of hardware circuitry and software.

The number and arrangement of components shown in FIG. 3 are provided as an example. In practice, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

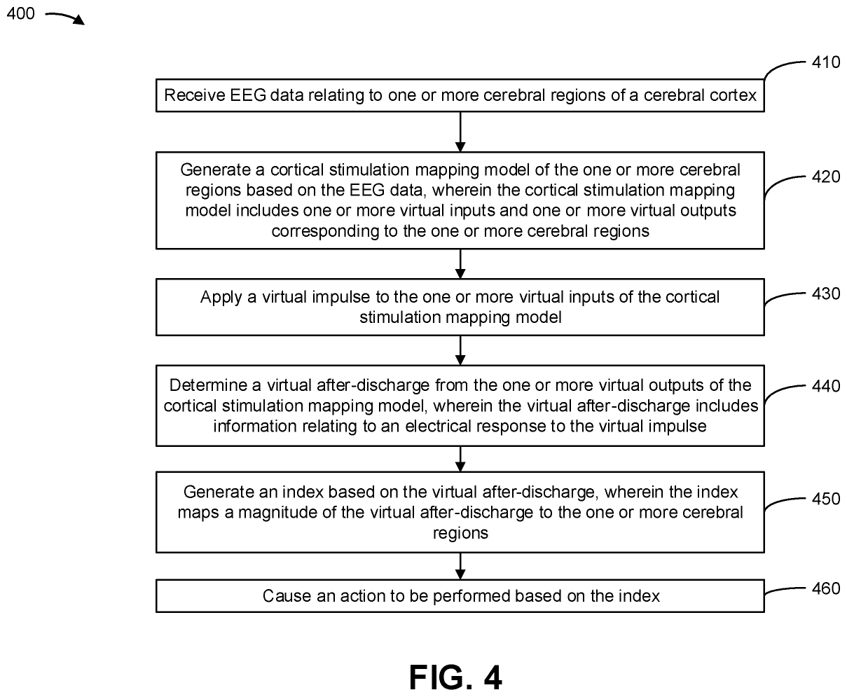

FIG. 4 is a flow chart of an example process 400 for localizing epileptogenic zones. In some implementations, one or more process blocks of FIG. 4 may be performed by a localization platform (e.g., localization platform 240). In some implementations, one or more process blocks of FIG. 4 may be performed by another device or a group of devices separate from or including the localization platform, such as a client device (e.g., client device 210), or a network storage device (e.g., network storage device 220).

As shown in FIG. 4, process 400 may include receiving EEG data relating to one or more cerebral regions of a cerebral cortex (block 410). For example, the localization platform (e.g., using computing resource 245, processor 320, memory 330, storage component 340, input component 350, output component 360, communication interface 370, and/or the like) may receive EEG data relating to one or more cerebral regions of a cerebral cortex, as described above.

As further shown in FIG. 4, process 400 may include generating, based on the EEG data, a cortical stimulation mapping model of the one or more cerebral regions, wherein the cortical stimulation mapping model includes one or more virtual inputs and one or more virtual outputs corresponding to the one or more cerebral regions (block 420). For example, the localization platform (e.g., using computing resource 245, processor 320, memory 330, storage component 340, input component 350, output component 360, communication interface 370, and/or the like) may generate, based on the EEG data, a cortical stimulation mapping model of the one or more cerebral regions, as described above. In some aspects, the cortical stimulation mapping model may include one or more virtual inputs and one or more virtual outputs corresponding to the one or more cerebral regions.

As further shown in FIG. 4, process 400 may include applying a virtual impulse to the one or more virtual inputs of the cortical stimulation mapping model (block 430). For example, the localization platform (e.g., using computing resource 245, processor 320, memory 330, storage component 340, input component 350, output component 360, communication interface 370, and/or the like) may apply a virtual impulse to the one or more virtual inputs of the cortical stimulation mapping model, as described above.

As further shown in FIG. 4, process 400 may include determining a virtual after-discharge from the one or more virtual outputs of the cortical stimulation mapping model, wherein the virtual after-discharge includes information relating to an electrical response to the virtual impulse (block 440). For example, the localization platform (e.g., using computing resource 245, processor 320, memory 330, storage component 340, input component 350, output component 360, communication interface 370, and/or the like) may determine a virtual after-discharge from the one or more virtual outputs of the cortical stimulation mapping model, as described above. In some aspects, the virtual after-discharge may include information relating to an electrical response to the virtual impulse.

As further shown in FIG. 4, process 400 may include generating an index based on the virtual after-discharge, wherein the index maps a magnitude of the virtual after-discharge to the one or more cerebral regions (block 450). For example, the localization platform (e.g., using computing resource 245, processor 320, memory 330, storage component 340, input component 350, output component 360, communication interface 370, and/or the like) may generate an index based on the virtual after-discharge, as described above. In some aspects, the index may map a magnitude of the virtual after-discharge to the one or more cerebral regions.

As further shown in FIG. 4, process 400 may include causing an action to be performed based on the index (block 460). For example, the localization platform (e.g., using computing resource 245, processor 320, memory 330, storage component 340, input component 350, output component 360, communication interface 370, and/or the like) may cause an action to be performed based on the index, as described above.

Process 400 may include additional implementations, such as any single implementation or any combination of implementations described below and/or in connection with one or more other processes described elsewhere herein.

In a first implementation, receiving the EEG data may comprise: receiving, from one of a set of network storage devices, one or more of ECoG data or SEEG data.

In a second implementation, alone or in combination with the first implementation, generating the cortical stimulation mapping model may comprise: generating the cortical stimulation mapping model based on a linear time varying network of the EEG data and using a least squares analysis.

In a third implementation, alone or in combination with one or more of the first and second implementations, generating the cortical stimulation mapping model may comprise: generating the cortical stimulation mapping model as a visual model of the one or more cerebral regions.

In a fourth implementation, alone or in combination with one or more of the first through third implementations, applying the virtual impulse may comprise: generating a unit impulse vector configured to cause a change in the magnitude of the virtual after-discharge; and applying the virtual impulse based on the unit impulse vector.

In a fifth implementation, alone or in combination with one or more of the first through fourth implementations, causing the action to be performed may comprise: comparing the magnitude of the virtual after-discharge with a threshold magnitude; and identifying an epileptogenic zone based on the magnitude of the virtual after-discharge and the threshold magnitude.

In a sixth implementation, alone or in combination with one or more of the first through fifth implementations, causing the action to be performed may comprise: determining an epileptogenicity of one of the one or more cerebral regions based on the index; generating a recommendation based on the epileptogenicity; and transmitting the recommendation to a client device.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

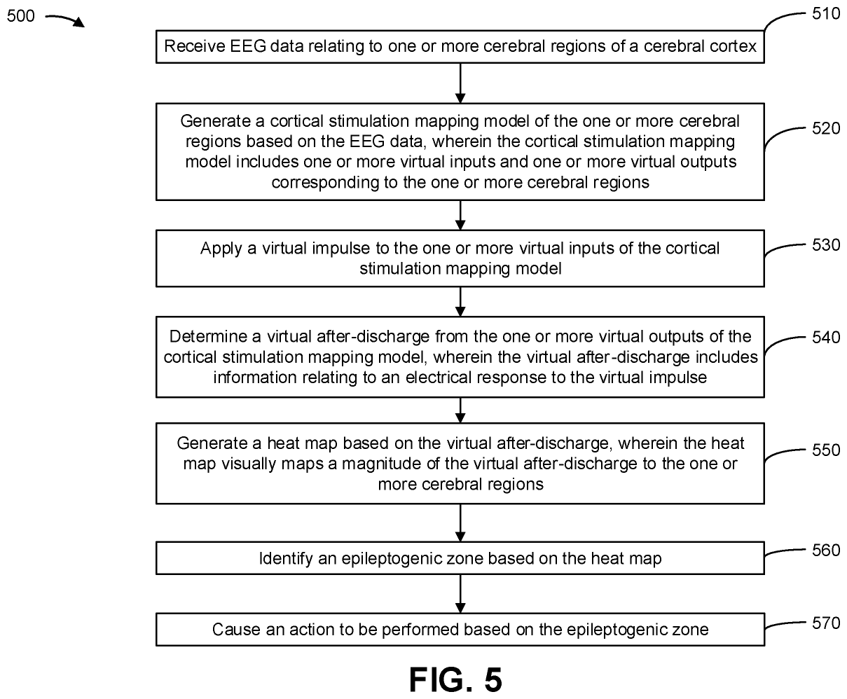

FIG. 5 is a flow chart of an example process 500 for localizing epileptogenic zones. In some implementations, one or more process blocks of FIG. 5 may be performed by a localization platform (e.g., localization platform 240). In some implementations, one or more process blocks of FIG. 5 may be performed by another device or a group of devices separate from or including the localization platform, such as a client device (e.g., client device 210), or a network storage device (e.g., network storage device 220).

As shown in FIG. 5, process 500 may include receiving EEG data relating to one or more cerebral regions of a cerebral cortex (block 510). For example, the localization platform (e.g., using computing resource 245, processor 320, memory 330, storage component 340, input component 350, output component 360, communication interface 370, and/or the like) may receive EEG data relating to one or more cerebral regions of a cerebral cortex, as described above.

As further shown in FIG. 5, process 500 may include generating a cortical stimulation mapping model of the one or more cerebral regions based on the EEG data, wherein the cortical stimulation mapping model includes one or more virtual inputs and one or more virtual outputs corresponding to the one or more cerebral regions (block 520). For example, the localization platform (e.g., using computing resource 245, processor 320, memory 330, storage component 340, input component 350, output component 360, communication interface 370, and/or the like) may generate a cortical stimulation mapping model of the one or more cerebral regions based on the EEG data, as described above. In some aspects, the cortical stimulation mapping model may include one or more virtual inputs and one or more virtual outputs corresponding to the one or more cerebral regions.

As further shown in FIG. 5, process 500 may include applying a virtual impulse to the one or more virtual inputs of the cortical stimulation mapping model (block 530). For example, the localization platform (e.g., using computing resource 245, processor 320, memory 330, storage component 340, input component 350, output component 360, communication interface 370, and/or the like) may apply a virtual impulse to the one or more virtual inputs of the cortical stimulation mapping model, as described above.

As further shown in FIG. 5, process 500 may include determining a virtual after-discharge from the one or more virtual outputs of the cortical stimulation mapping model, wherein the virtual after-discharge includes information relating to an electrical response to the virtual impulse (block 540). For example, the localization platform (e.g., using computing resource 245, processor 320, memory 330, storage component 340, input component 350, output component 360, communication interface 370, and/or the like) may determine a virtual after-discharge from the one or more virtual outputs of the cortical stimulation mapping model, as described above. In some aspects, the virtual after-discharge may include information relating to an electrical response to the virtual impulse.

As further shown in FIG. 5, process 500 may include generating a heat map based on the virtual after-discharge, wherein the heat map visually maps a magnitude of the virtual after-discharge to the one or more cerebral regions (block 550). For example, the localization platform (e.g., using computing resource 245, processor 320, memory 330, storage component 340, input component 350, output component 360, communication interface 370, and/or the like) may generate a heat map based on the virtual after-discharge, as described above. In some aspects, the heat map may visually map a magnitude of the virtual after-discharge to the one or more cerebral regions.

As further shown in FIG. 5, process 500 may include identifying an epileptogenic zone based on the heat map (block 560). For example, the localization platform (e.g., using computing resource 245, processor 320, memory 330, storage component 340, input component 350, output component 360, communication interface 370, and/or the like) may identify an epileptogenic zone based on the heat map, as described above.

As further shown in FIG. 5, process 500 may include causing an action to be performed based on the epileptogenic zone (block 570). For example, the localization platform (e.g., using computing resource 245, processor 320, memory 330, storage component 340, input component 350, output component 360, communication interface 370, and/or the like) may cause an action to be performed based on the epileptogenic zone, as described above.

Process 500 may include additional implementations, such as any single implementation or any combination of implementations described below and/or in connection with one or more other processes described elsewhere herein.

In a first implementation, applying the virtual impulse may comprise: generating a unit impulse vector configured to cause a change in the magnitude of the virtual after-discharge; and applying the virtual impulse based on the unit impulse vector.

In a second implementation, alone or in combination with the first implementation, generating the heat map may comprise: generating the heat map to include color-coded indications of the magnitude of the virtual after-discharge corresponding to the one or more cerebral regions.

In a third implementation, alone or in combination with one or more of the first and second implementations, identifying the epileptogenic zone may comprise: comparing the magnitude of the virtual after-discharge with a threshold magnitude; and identifying the epileptogenic zone based on the magnitude of the virtual after-discharge and the threshold magnitude. In some implementations, the epileptogenic zone may be determined to include one of the one or more cerebral regions based on determining that a magnitude of a virtual after-discharge corresponding to the one of the one or more cerebral regions satisfies the threshold magnitude, or the epileptogenic zone may be determined to not include the one of the one or more cerebral regions based on determining that the magnitude of the virtual after-discharge corresponding to the one of the one or more cerebral regions does not satisfy the threshold magnitude.

In a fourth implementation, alone or in combination with one or more of the first through third implementations, identifying the epileptogenic zone may comprise: determining an epileptogenicity of one of the one or more cerebral regions based on the heat map; generating a recommendation based on the epileptogenicity; and transmitting the recommendation to a client device.

In a fifth implementation, alone or in combination with one or more of the first through fourth implementations, causing the action to be performed may comprise: generating a visual model of the one or more cerebral regions; generating a graphical representation of one of the one or more cerebral regions based on the epileptogenic zone; overlaying the graphical representation of the one of the one or more cerebral regions on the visual model at a location corresponding to the one of the one or more cerebral regions; and transmitting the visual model to a client device. In some implementations, the graphical representation of the one of the one or more cerebral regions may be indicative of a magnitude of a virtual after-discharge corresponding to the one of the one or more cerebral regions.

In a sixth implementation, alone or in combination with one or more of the first through fifth implementations, the localization platform may further: receive, from a client device, a clinically annotated epileptogenic zone; compare the clinically annotated epileptogenic zone to the heat map; determine a treatment success rate based on the clinically annotated epileptogenic zone, generate a recommendation based on the treatment success rate; and transmit the recommendation to the client device. In some implementations, the treatment success rate may correspond to a likelihood that treatment of the clinically annotated epileptogenic zone will be successful in curing epilepsy of a subject.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

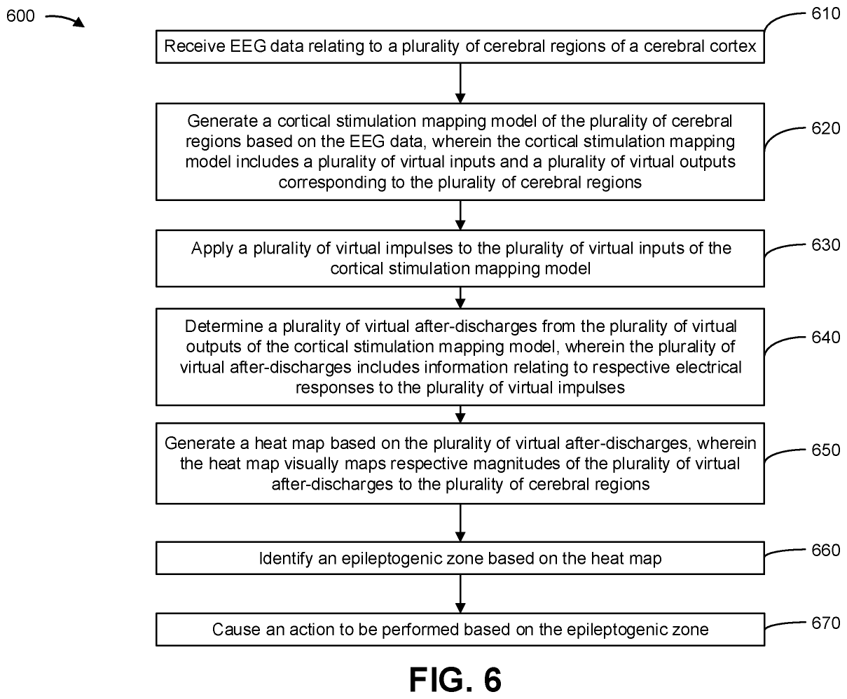

FIG. 6 is a flow chart of an example process 600 for localizing epileptogenic zones. In some implementations, one or more process blocks of FIG. 6 may be performed by a localization platform (e.g., localization platform 240). In some implementations, one or more process blocks of FIG. 6 may be performed by another device or a group of devices separate from or including the localization platform, such as a client device (e.g., client device 210), or a network storage device (e.g., network storage device 220).

As shown in FIG. 6, process 600 may include receiving EEG data relating to a plurality of cerebral regions of a cerebral cortex (block 610). For example, the localization platform (e.g., using computing resource 245, processor 320, memory 330, storage component 340, input component 350, output component 360, communication interface 370, and/or the like) may receive EEG data relating to a plurality of cerebral regions of a cerebral cortex, as described above.

As further shown in FIG. 6, process 600 may include generating a cortical stimulation mapping model of the plurality of cerebral regions based on the EEG data, wherein the cortical stimulation mapping model includes a plurality of virtual inputs and a plurality of virtual outputs corresponding to the plurality of cerebral regions (block 620). For example, the localization platform (e.g., using computing resource 245, processor 320, memory 330, storage component 340, input component 350, output component 360, communication interface 370, and/or the like) may generate a cortical stimulation mapping model of the plurality of cerebral regions based on the EEG data, as described above. In some aspects, the cortical stimulation mapping model may include a plurality of virtual inputs and a plurality of virtual outputs corresponding to the plurality of cerebral regions.

As further shown in FIG. 6, process 600 may include applying a plurality of virtual impulses to the plurality of virtual inputs of the cortical stimulation mapping model (block 630). For example, the localization platform (e.g., using computing resource 245, processor 320, memory 330, storage component 340, input component 350, output component 360, communication interface 370, and/or the like) may apply a plurality of virtual impulses to the plurality of virtual inputs of the cortical stimulation mapping model, as described above.

As further shown in FIG. 6, process 600 may include determining a plurality of virtual after-discharges from the plurality of virtual outputs of the cortical stimulation mapping model, wherein the plurality of virtual after-discharges includes information relating to respective electrical responses to the plurality of virtual impulses (block 640). For example, the localization platform (e.g., using computing resource 245, processor 320, memory 330, storage component 340, input component 350, output component 360, communication interface 370, and/or the like) may determine a plurality of virtual after-discharges from the plurality of virtual outputs of the cortical stimulation mapping model, as described above. In some aspects, the plurality of virtual after-discharges may include information relating to respective electrical responses to the plurality of virtual impulses.

As further shown in FIG. 6, process 600 may include generating a heat map based on the plurality of virtual after-discharges, wherein the heat map visually maps respective magnitudes of the plurality of virtual after-discharges to the plurality of cerebral regions (block 650). For example, the localization platform (e.g., using processor 320, memory 330, storage component 340, input component 350, output component 360, communication interface 370 and/or the like) may generate a heat map based on the plurality of virtual after-discharges, as described above. In some aspects, the heat map may visually map respective magnitudes of the plurality of virtual after-discharges to the plurality of cerebral regions.

As further shown in FIG. 6, process 600 may include identifying an epileptogenic zone based on the heat map (block 660). For example, the localization platform (e.g., using computing resource 245, processor 320, memory 330, storage component 340, input component 350, output component 360, communication interface 370, and/or the like) may identify an epileptogenic zone based on the heat map, as described above.

As further shown in FIG. 6, process 600 may include causing an action to be performed based on the epileptogenic zone (block 670). For example, the localization platform (e.g., using computing resource 245, processor 320, memory 330, storage component 340, input component 350, output component 360, communication interface 370, and/or the like) may cause an action to be performed based on the epileptogenic zone, as described above.

Process 600 may include additional implementations, such as any single implementation or any combination of implementations described below and/or in connection with one or more other processes described elsewhere herein.

In a first implementation, generating the cortical stimulation mapping model may comprise: generating the cortical stimulation mapping model as a visual model of the plurality of cerebral regions. In some implementations, the visual model may simulate an in-vivo cortical stimulation mapping procedure. In some implementations, the visual model may include a plurality of graphical representations of electrodes corresponding to the plurality of virtual inputs and the plurality of virtual outputs.

In a second implementation, alone or in combination with the first implementation, identifying the epileptogenic zone may comprise: comparing the respective magnitudes of the plurality of virtual after-discharges with a threshold magnitude; and identifying the epileptogenic zone based on the respective magnitudes of the plurality of virtual after-discharges and the threshold magnitude. In some implementations, the epileptogenic zone may be determined to include an area associated with one of the plurality of cerebral regions based on determining that a respective magnitude of a virtual after-discharge corresponding to the one of the plurality of cerebral regions satisfies the threshold magnitude, or the epileptogenic zone may be determined to not include an area associated with the one of the plurality of cerebral regions based on determining that the respective magnitude of the virtual after-discharge corresponding to the one of the plurality of cerebral regions does not satisfy the threshold magnitude.

In a third implementation, alone or in combination with one or more of the first and second implementations, identifying the epileptogenic zone may comprise: determining an epileptogenicity of one of the plurality of cerebral regions based on the heat map; generating a recommendation based on the epileptogenicity; and transmitting the recommendation to a client device.

In a fourth implementation, alone or in combination with one or more of the first through third implementations, causing the action to be performed may comprise: generating a visual model of the plurality of cerebral regions; generating a graphical representation of one of the plurality of cerebral regions based on the epileptogenic zone; overlaying the graphical representation of the one of the plurality of cerebral regions on the visual model at a location corresponding to the one of the plurality of cerebral regions; and transmitting the visual model to a client device. In some implementations, the graphical representation of the one of the plurality of cerebral regions may be indicative of a respective magnitude of a virtual after-discharge corresponding to the one of the plurality of cerebral regions.

In a fifth implementation, alone or in combination with one or more of the first through fourth implementations, the localization platform may further: receive, from a client device, a clinically annotated epileptogenic zone; compare the clinically annotated epileptogenic zone to the heat map; determine a treatment success rate based on the clinically annotated epileptogenic zone; generate a recommendation based on the treatment success rate; and transmit the recommendation to the client device. In some implementations, the treatment success rate may correspond to a likelihood that treatment of the clinically annotated epileptogenic zone will be successful in curing epilepsy of a subject.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

The foregoing disclosure provides illustration and description, but is not intended to be exhaustive or to limit the implementations to the precise form disclosed. Modifications and variations may be made in light of the above disclosure or may be acquired from practice of the implementations.

As used herein, the term “component” is intended to be broadly construed as hardware, firmware, or a combination of hardware and software.

Some implementations are described herein in connection with thresholds. As used herein, satisfying a threshold may, depending on the context, refer to a value being greater than the threshold, more than the threshold, higher than the threshold, greater than or equal to the threshold, less than the threshold, fewer than the threshold, lower than the threshold, less than or equal to the threshold, equal to the threshold, and/or the like, depending on the context.

Certain user interfaces have been described herein and/or shown in the figures. A user interface may include a graphical user interface, a non-graphical user interface, a text-based user interface, and/or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, and/or the like). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

To the extent the aforementioned implementations collect, store, or employ personal information of individuals, it should be understood that such information shall be used in accordance with all applicable laws concerning protection of personal information. Additionally, the collection, storage, and use of such information can be subject to consent of the individual to such activity, for example, through well known “opt-in” or “opt-out” processes as can be appropriate for the situation and type of information. Storage and use of personal information can be in an appropriately secure manner reflective of the type of information, for example, through various encryption and anonymization techniques for particularly sensitive information.

It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method, comprising:
receiving, by a device, electroencephalography data relating to one or more cerebral regions of a cerebral cortex;
generating, by the device, and based on the electroencephalography data, a cortical stimulation mapping model of the one or more cerebral regions,
wherein the cortical stimulation mapping model includes one or more virtual inputs and one or more virtual outputs corresponding to the one or more cerebral regions;
applying, by the device, a virtual impulse to the one or more virtual inputs of the cortical stimulation mapping model;
determining, by the device, a virtual after-discharge from the one or more virtual outputs of the cortical stimulation mapping model,
wherein the virtual after-discharge includes information relating to an electrical response to the virtual impulse;
generating, by the device, an index based on the virtual after-discharge,
wherein the index maps a magnitude of the virtual after-discharge to the one or more cerebral regions; and
causing, by the device, an action to be performed based on the index.

2. The method of claim 1, wherein receiving the electroencephalography data comprises:
receiving, from one of a set of network storage devices, one or more of electrocorticography data or stereo-electroencephalography data.

3. The method of claim 1, wherein generating the cortical stimulation mapping model comprises:
generating the cortical stimulation mapping model based on a linear time varying network of the electroencephalography data and using a least squares analysis.

4. The method of claim 1, wherein generating the cortical stimulation mapping model comprises:
generating the cortical stimulation mapping model as a visual model of the one or more cerebral regions,
wherein the visual model simulates an in-vivo cortical stimulation mapping procedure, and
wherein the visual model includes one or more graphical representations of electrodes corresponding to the one or more virtual inputs and the one or more virtual outputs.

5. The method of claim 1, wherein applying the virtual impulse comprises:
generating a unit impulse vector configured to cause a change in the magnitude of the virtual after-discharge; and
applying the virtual impulse based on the unit impulse vector.

6. The method of claim 1, wherein causing the action to be performed comprises:
comparing the magnitude of the virtual after-discharge with a threshold magnitude; and
identifying an epileptogenic zone based on the magnitude of the virtual after-discharge and the threshold magnitude,
wherein the epileptogenic zone is determined to include an area associated with one of the one or more cerebral regions based on determining that a magnitude of a virtual after-discharge corresponding to the one of the one or more cerebral regions satisfies the threshold magnitude, or
wherein the epileptogenic zone is determined to not include an area associated with the one of the one or more cerebral regions based on determining that the magnitude of the virtual after-discharge corresponding to the one of the one or more cerebral regions does not satisfy the threshold magnitude.

7. The method of claim 1, wherein causing the action to be performed comprises:
determining an epileptogenicity of one of the one or more cerebral regions based on the index;
generating a recommendation based on the epileptogenicity; and
transmitting the recommendation to a client device.

8. A device, comprising:
one or more memories; and
one or more processors, communicatively coupled to the one or more memories, to:
receive electroencephalography data relating to one or more cerebral regions of a cerebral cortex;
generate a cortical stimulation mapping model of the one or more cerebral regions based on the electroencephalography data,
wherein the cortical stimulation mapping model includes one or more virtual inputs and one or more virtual outputs corresponding to the one or more cerebral regions;
apply a virtual impulse to the one or more virtual inputs of the cortical stimulation mapping model;
determine a virtual after-discharge from the one or more virtual outputs of the cortical stimulation mapping model,
wherein the virtual after-discharge includes information relating to an electrical response to the virtual impulse;
generate a heat map based on the virtual after-discharge,
wherein the heat map visually maps a magnitude of the virtual after-discharge to the one or more cerebral regions;
identify an epileptogenic zone based on the heat map; and
cause an action to be performed based on the epileptogenic zone.

9. The device of claim 8, wherein the one or more processors, when applying the virtual impulse, are to:
generating a unit impulse vector configured to cause a change in the magnitude of the virtual after-discharge; and
applying the virtual impulse based on the unit impulse vector.

10. The device of claim 8, wherein the one or more processors, when generating the heat map, are to:
generate the heat map to include color-coded indications of the magnitude of the virtual after-discharge corresponding to the one or more cerebral regions.

11. The device of claim 8, wherein the one or more processors, when identifying the epileptogenic zone, are to:
compare the magnitude of the virtual after-discharge with a threshold magnitude; and
identify the epileptogenic zone based on the magnitude of the virtual after-discharge and the threshold magnitude,
wherein the epileptogenic zone is determined to include one of the one or more cerebral regions based on determining that a magnitude of a virtual after-discharge corresponding to the one of the one or more cerebral regions satisfies the threshold magnitude, or
wherein the epileptogenic zone is determined to not include the one of the one or more cerebral regions based on determining that the magnitude of the virtual after-discharge corresponding to the one of the one or more cerebral regions does not satisfy the threshold magnitude.

12. The device of claim 8, wherein the one or more processors, when identifying the epileptogenic zone, are to:
determine an epileptogenicity of one of the one or more cerebral regions based on the heat map;
generate a recommendation based on the epileptogenicity; and
transmit the recommendation to a client device.

13. The device of claim 8, wherein the one or more processors, when causing the action to be performed, are to:
generate a visual model of the one or more cerebral regions; and
generate a graphical representation of one of the one or more cerebral regions based on the epileptogenic zone,
wherein the graphical representation of the one of the one or more cerebral regions is indicative of a magnitude of a virtual after-discharge corresponding to the one of the one or more cerebral regions;
overlay the graphical representation of the one of the one or more cerebral regions on the visual model at a location corresponding to the one of the one or more cerebral regions; and
transmit the visual model to a client device.

14. The device of claim 8, wherein the one or more processors are further to:
receive, from a client device, a clinically annotated epileptogenic zone;
compare the clinically annotated epileptogenic zone to the heat map;
determine a treatment success rate based on the clinically annotated epileptogenic zone,
wherein the treatment success rate corresponds to a likelihood that treatment of the clinically annotated epileptogenic zone will be successful in curing epilepsy of a subject;
generate a recommendation based on the treatment success rate; and
transmit the recommendation to the client device.

15. A non-transitory computer-readable medium storing instructions, the instructions comprising:
one or more instructions that, when executed by one or more processors, cause the one or more processors to:
receive electroencephalography data relating to a plurality of cerebral regions of a cerebral cortex;
generate a cortical stimulation mapping model of the plurality of cerebral regions based on the electroencephalography data,
wherein the cortical stimulation mapping model includes a plurality of virtual inputs and a plurality of virtual outputs corresponding to the plurality of cerebral regions;
apply a plurality of virtual impulses to the plurality of virtual inputs of the cortical stimulation mapping model;
determine a plurality of virtual after-discharges from the plurality of virtual outputs of the cortical stimulation mapping model,
wherein the plurality of virtual after-discharges includes information relating to respective electrical responses to the plurality of virtual impulses;
generate a heat map based on the plurality of virtual after-discharges,
wherein the heat map visually maps respective magnitudes of the plurality of virtual after-discharges to the plurality of cerebral regions;
identify an epileptogenic zone based on the heat map; and
cause an action to be performed based on the epileptogenic zone.

16. The non-transitory computer-readable medium of claim 15, wherein the one or more instructions, that cause the one or more processors to generate the cortical stimulation mapping model, cause the one or more processors to:
generate the cortical stimulation mapping model as a visual model of the plurality of cerebral regions,
wherein the visual model simulates an in-vivo cortical stimulation mapping procedure, and
wherein the visual model includes a plurality of graphical representations of electrodes corresponding to the plurality of virtual inputs and the plurality of virtual outputs.

17. The non-transitory computer-readable medium of claim 15, wherein the one or more instructions, that cause the one or more processors to identify the epileptogenic zone, cause the one or more processors to:
compare the respective magnitudes of the plurality of virtual after-discharges with a threshold magnitude; and
identify the epileptogenic zone based on the respective magnitudes of the plurality of virtual after-discharges and the threshold magnitude,
wherein the epileptogenic zone is determined to include an area associated with one of the plurality of cerebral regions based on determining that a respective magnitude of a virtual after-discharge corresponding to the one of the plurality of cerebral regions satisfies the threshold magnitude, or
wherein the epileptogenic zone is determined to not include an area associated with the one of the plurality of cerebral regions based on determining that the respective magnitude of the virtual after-discharge corresponding to the one of the plurality of cerebral regions does not satisfy the threshold magnitude.

18. The non-transitory computer-readable medium of claim 15, wherein the one or more instructions, that cause the one or more processors to identify the epileptogenic zone, cause the one or more processors to:
determine an epileptogenicity of one of the plurality of cerebral regions based on the heat map;
generate a recommendation based on the epileptogenicity; and
transmit the recommendation to a client device.

19. The non-transitory computer-readable medium of claim 15, wherein the one or more instructions, that cause the one or more processors to cause the action to be performed, cause the one or more processors to:
generate a visual model of the plurality of cerebral regions; and
generate a graphical representation of one of the plurality of cerebral regions based on the epileptogenic zone,
wherein the graphical representation of the one of the plurality of cerebral regions is indicative of a respective magnitude of a virtual after-discharge corresponding to the one of the plurality of cerebral regions;
overlay the graphical representation of the one of the plurality of cerebral regions on the visual model at a location corresponding to the one of the plurality of cerebral regions; and
transmit the visual model to a client device.

20. The non-transitory computer-readable medium of claim 15, wherein the one or more instructions, when executed by the one or more processors, further cause the one or more processors to:
receive, from a client device, a clinically annotated epileptogenic zone;
compare the clinically annotated epileptogenic zone to the heat map;
determine a treatment success rate based on the clinically annotated epileptogenic zone,
wherein the treatment success rate corresponds to a likelihood that treatment of the clinically annotated epileptogenic zone will be successful in curing epilepsy of a subject;
generate a recommendation based on the treatment success rate; and
transmit the recommendation to the client device.
